(19) United States
(12) Patent Application Publication
(10) Pub. No.: US 2006/0217635 A1
(43) Pub. Date: Sep. 28, 2006

(54) Biopsy device marker deployment

(76) Inventors: Elizabeth S. McCombs, Cincinnati, OH (US); Shawn C. Synder, Greendale, IN (US); Wells D. Haberstich, (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/370,357

(22) Filed: Mar. 8, 2006

Related U.S. Application Data
(60) Provisional application No. 60/665,432, filed on Mar. 24, 2005.

(51) Int. Cl. A61B 10/00

(52) U.S. Cl. 600/566; 600/567

(57) ABSTRACT

A biopsy device having a cannula and a cutter is disclosed. The biopsy device includes a guide, such as guide passageway formed in a portion of the biopsy device. The guide passageway assists in positioning a marker deployer in a cutter lumen of the cannula.

This application cross references and incorporates by reference U.S. patent application Ser. No. 10/785,755 filed Feb. 24, 2004 “Biopsy Device with Variable Speed Cutter Advance.”

### FIELD OF THE INVENTION

The present invention relates in general to biopsy devices and biopsy markers, and more particularly to deployment of biopsy markers.

### BACKGROUND OF THE INVENTION

The diagnosis and treatment of patients with cancerous tumors is an ongoing area of investigation. Medical devices for obtaining tissue samples for subsequent sampling are known in the art. For instance, a biopsy instrument now marketed under the tradename MAMMOTOME is commercially available for use in obtaining breast biopsy samples.

The following patent documents disclose various biopsy devices and are incorporated herein by reference in their entirety: U.S. Pat. No. 6,273,862 issued Aug. 14, 2001; U.S. Pat. No. 6,231,522 issued May 15, 2001; U.S. Pat. No. 6,228,055 issued May 8, 2001; U.S. Pat. No. 6,120,462 issued Sep. 19, 2000; U.S. Pat. No. 6,086,544 issued Jul. 11, 2000; U.S. Pat. No. 6,077,230 issued Jun. 20, 2000; U.S. Pat. No. 6,017,316 issued Jan. 25, 2000; U.S. Pat. No. 6,007,497 issued Dec. 28, 1999; U.S. Pat. No. 5,980,469 issued Nov. 9, 1999; U.S. Pat. No. 5,964,716 issued Oct. 12, 1999; U.S. Pat. No. 5,928,164 issued Jul. 27, 1999; U.S. Pat. No. 5,775,333 issued Jul. 7, 1998; U.S. Pat. No. 5,769,086 issued Jun. 23, 1998; U.S. Pat. No. 5,649,547 issued Jul. 22, 1997; U.S. Pat. No. 5,526,822 issued Jun. 18, 1996; US 2003/0199785 published Oct. 23, 2003; US 2003/0199754 published Oct. 23, 2003; US 2003/0199754 published Oct. 23, 2003.

Biopsy markers for marking biopsy sites are known in the art. The following patent documents disclose biopsy markers and/or devices for deploying biopsy markers, and are incorporated herein by reference in their entirety:

U.S. Pat. No. 5,941,890 issued Aug. 24, 1999; U.S. Pat. No. 6,228,055 issued May 8, 2001; U.S. Pat. No. 6,261,302 issued Jul. 17, 2001; U.S. Pat. No. 6,356,782 issued Mar. 12, 2002; and U.S. Pat. No. 6,270,464 issued Aug. 7, 2001.

### SUMMARY OF THE INVENTION

Applicant's have recognized the desirability of providing a guide passageway in a biopsy device for simplifying the use of biopsy marker deployers, such as by assisting in the insertion of a biopsy marker deployer into an open proximal end of a cutter lumen in a hollow cannula of the biopsy device.

In one embodiment, the invention provides a biopsy device comprising a cannula having longitudinal axis, a cutter lumen, and a tissue receiving port communicating with the cutter lumen; a tissue cutter adapted for translation within the cutter lumen to sever tissue received in the tissue receiving port of the cannula; and at least one guide disposed proximal of the cannula for positioning a biopsy marker deployer with respect to the cutter lumen. In one embodiment, two guide passages are provided, one on either side of the biopsy device. Each guide passage can communicate with a non-circular side port opening on an outer surface of the biopsy device.

The present invention can also provide a method for deploying a biopsy marker. The method can include the steps of: providing a cannula having a longitudinal axis, a cutter lumen, and a tissue receiving port communicating with the cutter lumen; providing a tissue cutter adapted for translation within the cutter lumen to sever tissue received in the tissue receiving port of the cannula; providing a marker deployer adapted to deliver at least one biopsy marker; positioning the tissue receiving port of the cannula in tissue to be sampled; advancing the tissue cutter distally within the cutter lumen to sever a tissue sample; withdrawing the tissue cutter from the cutter lumen; inserting a portion of the marker deployer through a guide angled with respect to the longitudinal axis of the cannula; positioning a distal end of the marker deployer in the cutter lumen; and deploying a biopsy marker.

### BRIEF DESCRIPTION OF THE DRAWINGS

While the specification concludes with claims particularly pointing out and distinctly claiming the present invention, it is believed the same will be better understood by reference to the following description, taken in conjunction with the accompanying drawings in which:

FIG. 1 is an isometric view of a biopsy instrument having side access ports according to one embodiment of the present invention, and showing the internal cutter retracted to a proximal position;

FIG. 2 is an isometric illustration of the instrument of FIG. 1, showing the internal cutter advanced into an outer cannula, with the distal cutting end of the internal cutter visible through a lateral tissue receiving port in the outer cannula.

FIG. 3 is an isometric illustration of the biopsy instrument of FIG. 1 showing a marker deployment device positioned for insertion into a side access port in the biopsy instrument.

FIG. 4 is an isometric illustration of the biopsy instrument of FIG. 1 showing the marker deployment device inserted into the side access port of the biopsy instrument such that a proximal portion of a flexible deployer tube extends through a guide passageway in the body of the biopsy instrument communicating with the side access port, and such that the distal end of the flexible deployer tube is positioned to deploy a marker through the lateral tissue access port in the outer cannula.

FIG. 5 is a schematic top view illustration of a biopsy instrument of the type shown in FIG. 1 with a portion of the outer cannula piercing member cut away to reveal a cutter lumen and with guide passageways (shown in phantom) inclined with respect to a longitudinal axis of the outer cannula.

### DETAILED DESCRIPTION OF THE INVENTION

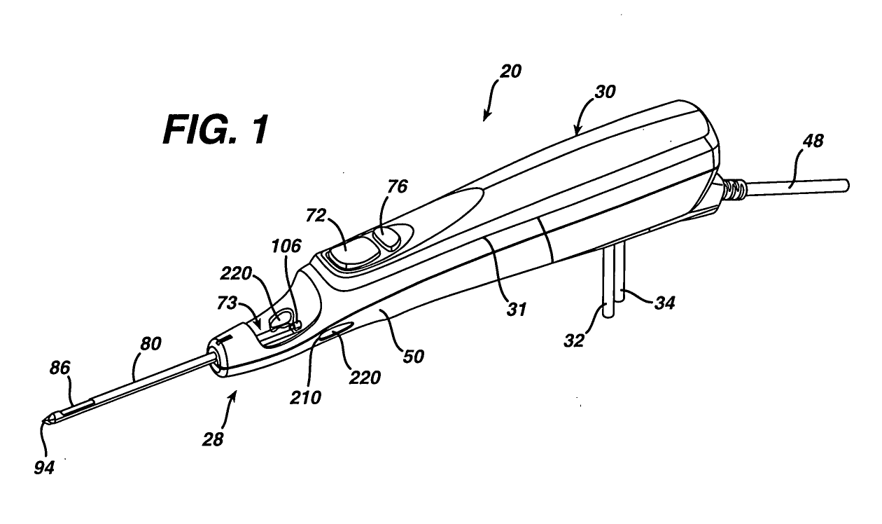

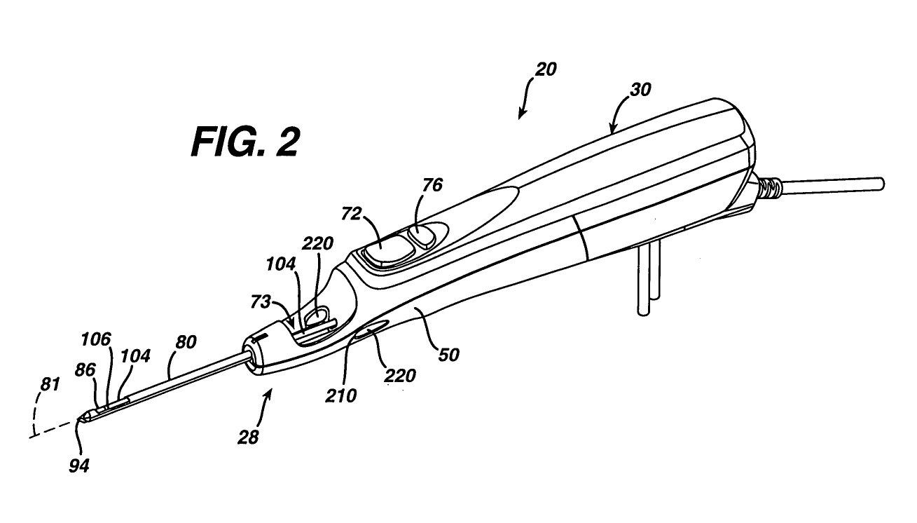

Referring to FIGS. 1 and 2, a biopsy instrument according to one embodiment of the present invention comprises a handpiece identified generally by the numeral 20. Handpiece 20 is preferably lightweight and ergonomically-shaped to be easily manipulated by an operator's hand. Handpiece 20 includes a probe assembly 28 and a detachably connected holster 30. Probe assembly 28 can be detachable from holster 30 along interface 31. Probe assembly 28 can be connected to a vacuum system, such as by first vacuum tube 32 and second vacuum tube 34. Holster 30 can include a control cord 48 operationally connecting the handpiece 20 to a control unit and power source.

Because handpiece 20 is manipulated by the operator's hand rather than by an electromechanical arm, the operator may steer the tip of handpiece 20 with great freedom towards the tissue mass of interest. The surgeon has tactile feedback while doing so and can thus ascertain, to a significant degree, the density and hardness of the tissue being encountered. In addition, handpiece 20 may be held approximately parallel to the chest wall of the patient for obtaining tissue portions closer to the chest wall than may be obtained when using an instrument mounted to an electromechanical arm. Those skilled in the art may appreciate that a mount or “nest” could be provided to hold handpiece 20 securely to the movable arm of an X-ray stereotactic table in the event that it is desirable to use an X-ray stereotactic table.

Holster 30 can include one or more switches to enable the operator to use the handpiece 20 with a single hand. These switches can include a rocker switch 72 for actuating the motion of a cutter (such as hollow tubular cutter 104) and a vacuum switch 76 for actuating a vacuum system. One-handed operation allows the operator's other hand to be free, for example, to hold an ultrasonic imaging device, or to deploy a biopsy marker to mark a biopsy site.

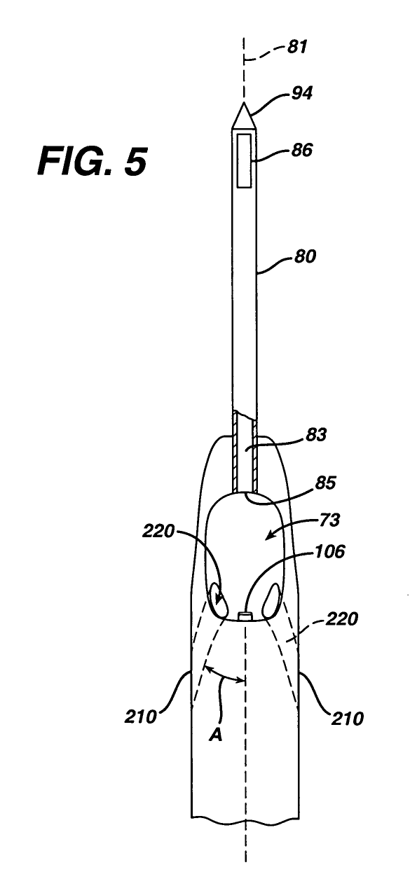

Probe assembly 28 can include a body including an outer shell 50. Outer shell 50 can be formed of one or more segments which may be injection molded from a rigid, biocompatible plastic, such as a polycarbonate. The outer shell 50 can be shaped to define a recess 73 for retrieving tissue samples extracted by probe assembly 28. Probe assembly 28 can include a hollow outer cannula piercing member 80 extending distally from the outer shell 50. The piercing member 80 can have a distal tissue piercing tip 94, an internal cutter lumen 83 (shown in FIG. 5), and a lateral tissue receiving port 86 communicating with the internal cutter lumen 83. The piercing member 80 can extend along a longitudinal axis 81, as shown in FIG. 2. The piercing member 80 can have a proximal end supported at the distal end of shell 50. The cutter lumen 83 of piercing member 80 can be open to the tissue sample retrieval recess 73 at the proximal end of member 80. Probe assembly can include an inner cutter 104 which is adapted for translation and rotation within the cutter lumen of member 80, as described in above referenced U.S. patent Ser. No. 10/785,755.

To obtain a biopsy sample, the port 86 of the piercing member 80 can be positioned in tissue, such as by piercing exterior skin with the tip 94 of piercing member 80. An internal cutter 104 can be retracted from the cutter lumen of member 80, thereby permitting tissue to be received in the cutter lumen 83 through port 86. The cutter 104 can be retracted to a position proximal of the recess 73, such as shown in FIG. 1 and FIG. 5. A tissue mass can then be received in the cutter lumen 83 of the piercing member 80 through port 86 (such as with vacuum assist). Hollow tubular cutter 104 can then be advanced from a proximal end of recess 73 to traverse across recess 73 and into the cutter lumen to sever a tissue sample from the tissue mass received in port 86, with the cutter 104 translating and rotating as the cutter advances past port 86. In FIG. 1, the distal end of cutter 104 is shown positioned at a proximal end of the recess 73. In FIG. 2, the cutter 104 is shown in a position where cutter 104 has been advanced across the length of recess 73 and into the cutter lumen 83 of member 80, with the distal end 106 of cutter 104 being visible as it traverses the length of port 86.

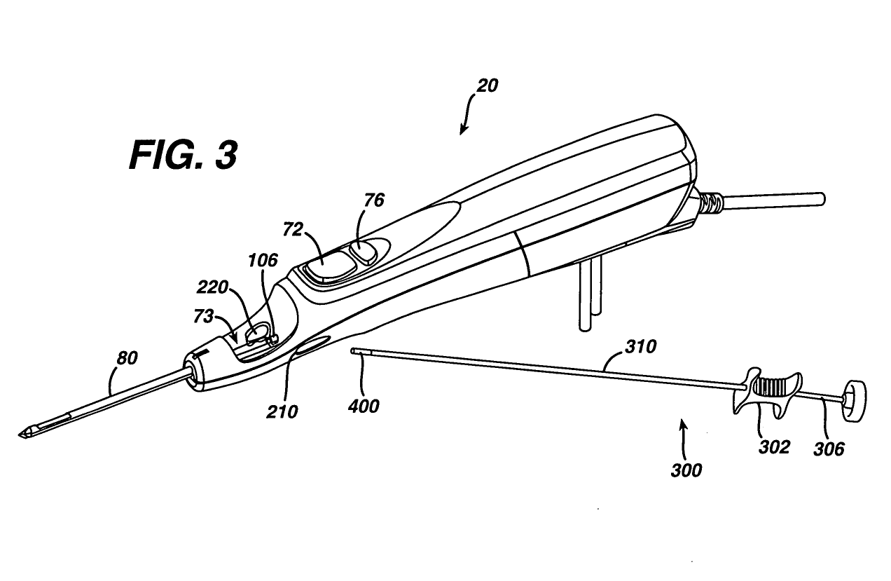

Once a tissue sample has been severed by cutter 104, the sample can be transported proximally (such as by cutter 104) and deposited in recess 73. It may then be desirable to place a biopsy marker at the biopsy site prior to withdrawing the piercing member 80 from the patient. In FIG. 3, the cutter 104 is shown retracted after severing of a tissue sample, with the distal end 106 of cutter 104 retracted to a position at the proximal end of recess 73.

FIG. 3 also illustrates a biopsy marker deployer 300. The biopsy marker deployer 300 can include, for example, a base 302, a flexible shaft 310 (which can be hollow) extending from base 302, and a rod 306 advancable in flexible shaft 310. A marker element 400 can be disposed at, or within, the distal end of the flexible shaft 310. Marker 400 can be deployed from deployer 300, such as by advancement of rod 306 in flexible shaft 310.

According to the present invention, one or more guides are provided to assist in positioning a marker deployer, such as deployer 300, in the open proximal end of the cutter lumen 83 of piercing member 80. In FIGS. 1-5, the outer shell 50 is shown to include one or more side ports 210. Each side port 210 can be positioned proximally of the open proximal end 85 of cutter lumen 83 in the piercing member 80, and can be offset laterally from the axis 81 of the piercing member 81 (the lateral direction being perpendicular to the direction of axis 81 in FIG. 5). Each side port 210 can communicate with a guide for receiving a portion of a marker deployer. In the embodiments shown, the guides are in the form of guide passages 220. Alternatively, the guides can be in the form of grooves or channels, such as grooves or channels formed in or associated with the outer surface of the probe assembly 28. In FIGS. 1-5, two guide passages 220 are illustrated, one on each side of the recess 73 to facilitate insertion of a marker deployer 300 from either the left hand or right hand side of the biopsy instrument 20.

As shown in FIGS. 1-4, the ports 210 can have a non-circular, elongated shape. Each guide passage 220 can extend distally and laterally inwardly from its associated port 210. FIG. 5 illustrates the guide passages 220 in phantom. Each guide passage 220 can extend through a portion of the body of the probe assembly 28, and can be formed in the shell 50 or other portion of the body of the probe assembly. Alternatively, the passages 220 can be formed in a separate structure or insert received in, or supported by, the probe assembly 28.

The guide passages 220 can be sized to receive the flexible shaft 310 of a biopsy marker deployer 300. Each guide passage 220 can be generally straight, or alternatively can be curved, and each passage 220 can extend from side port 210 to communicate with the recess 73. The guide passages can be angled with respect to axis 81 of piercing member 80 to assist in guiding the flexible shaft 310 into the open proximal end of the cutter lumen in piercing member 80. The guide passages 220 can be oriented with respect to the piercing element 80 to form included angle A (FIG. 5) with the axis 81 of less than about 60 degrees, in one embodiment less than about 45 degrees, and more particularly less than about 30 degrees. The longitudinal axis 81 and the axes of the guide passages 220 can lie in substantially the same plane. In one embodiment, the guide passages provide line of site access to the open proximal end 85 of the cutter lumen, in that the open proximal end 85 of the cutter lumen is visible viewed through the side ports 210.

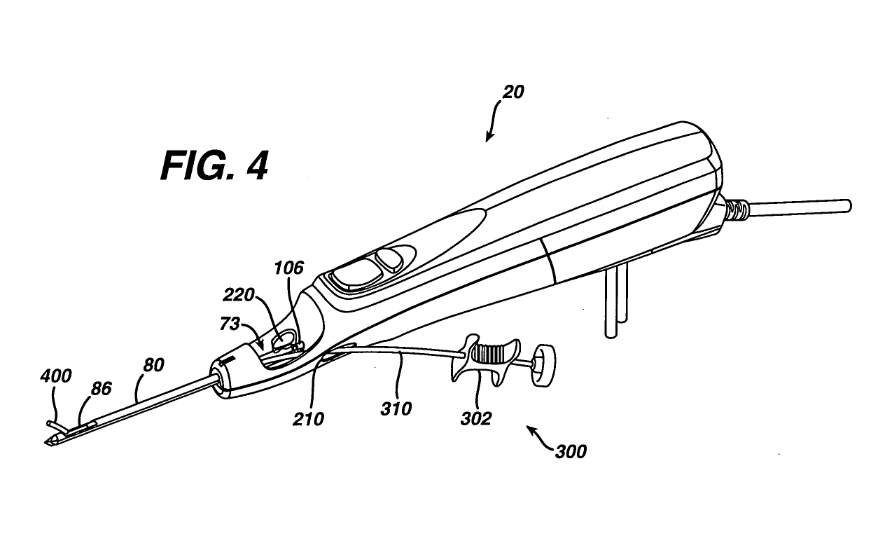

In FIG. 4, with cutter 104 retracted proximally, the shaft 310 of marker deployer 300 is shown inserted in port 210, through guide passage 220, and into the cutter lumen of piercing member 80. The distal end of shaft 310 can be positioned at or near port 86 of piercing member 80, and the marker deployer 300 can be activated to deploy the marker 400 through the port 86, as illustrated in FIG. 4.

Once the marker 400 has been deployed to mark the biopsy site, the shaft 310 can be removed from the cutter lumen in piercing member 80. If desired, the port 86 can be repositioned in the patient's tissue, additional biopsy samples can be taken, and additional markers can be deployed with assistance of guide passages 220.

While embodiment of the present invention have been shown and described herein, those skilled in the art will recognize that such embodiments are provided by way of example, and that numerous variations, changes, and substitutions will now occur to those skilled in the art without departing from the spirit and scope of the present invention. Further, each element disclosed may be alternatively described as a means for performing the element's function.

What is claimed:

1. A biopsy device comprising:
a cannula having longitudinal axis, a cutter lumen, and a tissue receiving port communicating with the cutter lumen;
a tissue cutter adapted for translation within the cutter lumen to sever tissue received in the tissue receiving port of the cannula; and
at least one guide disposed proximal of the cannula for positioning a biopsy marker deployer with respect to the cutter lumen.

2. The biopsy device of claim 1 wherein the guide is laterally offset from a longitudinal axis of the cannula.

3. The biopsy device of claim 1 wherein the guide comprises a passageway.

4. The biopsy device of claim 1 wherein the guide forms an included angle with the longitudinal axis of the cannula of less than 60 degrees.

5. The biopsy device of claim 1 wherein the guide comprises a guide side port in an outer surface of the biopsy device.

6. The biopsy device of claim 5 wherein the side port is non-circular.

7. The biopsy device of claim 1 comprising at least two guides.

8. The biopsy device of claim 1 wherein the guide is spaced laterally and longitudinally from the cannuala.

9. A biopsy device comprising:
a body;
a cannula extending distally from the body, the cannula having longitudinal axis, a cutter lumen, and a tissue receiving port communicating with the cutter lumen;
a tissue cutter adapted for translation within the cutter lumen to sever tissue received in the tissue receiving port of the cannula; and
at least one guide passageway disposed proximal of the cannula, wherein the guide passageway is angled with respect to the longitudinal axis of the cannula.

10. The biopsy device of claim 9 wherein the guide passageway comprises a side port.

11. The biopsy device of claim 10 wherein the side port is non-circular.

12. A biopsy method comprising the steps of:
providing a cannula having longitudinal axis, a cutter lumen, and a tissue receiving port communicating with the cutter lumen;
providing a tissue cutter adapted for translation within the cutter lumen to sever tissue received in the tissue receiving port of the cannula;
providing a marker deployer adapted to deliver at least one biopsy marker;
positioning the tissue receiving port of the cannula in tissue to be sampled;
advancing the tissue cutter distally within the cutter lumen to sever a tissue sample;
withdrawing the tissue cutter from the cutter lumen;
inserting a portion of the marker deployer through a guide angled with respect to the longitudinal axis of the cannula;
positioning a distal end of the marker deployer in the cutter lumen; and
deploying a biopsy marker.

13. The method of claim 12 wherein the step of inserting a portion of the marker deployer comprises inserting a portion of the marker deployer through a passageway in a portion of a biopsy device.

14. The method of claim 13 wherein the distal end of the marker deployer exits the passageway prior to entering the cutter lumen.
